(19) United States
(12) Patent Application Publication
(10) Pub. No.: US 2007/0071729 A1
(43) Pub. Date: Mar. 29, 2007

(54) Dermal fillers for biomedical applications in mammals and methods of using the same

(76) Inventor: Eric F. Bernstein, Gladwyne, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/409,024

(22) Filed: Apr. 24, 2006

Related U.S. Application Data
(60) Provisional application No. 60/674,314, filed on Apr. 25, 2005.

(51) Int. Cl. A61K 35/36

(52) U.S. Cl. 424/93.7; 424/401

(57) ABSTRACT

A method of tissue augmentation in mammals comprising implanting or injecting into the tissue of a mammal elastic material derived from solar elastosis or other sun-damaged sites in humans is disclosed. The method further comprises injecting into the tissue of a mammal, alone, or in combination with the solar elastosis, at least one filler material, such as fibrillin, recombinant fibrillin, fibulin, and recombinant fibulin. A method of treating various skin conditions, such as wrinkles, photoaging, acne scarring, scars, and HIV-associated lipoatrophy, by injecting into the tissue elastic material derived from solar elastosis and/or filler material is also disclosed.

This application claims the benefit of domestic priority to U.S. Provisional Patent Application Ser. No. 60/674,314, filed Apr. 25, 2005, which is herein incorporated by reference in its entirety.

The present disclosure is related to dermal fillers for biomedical applications in humans and animals, such as elastic material derived from solar elastosis or other sun-damaged sites in humans, or filler materials, such as fibrillin, recombinant fibrillin, fibulin, and recombinant fibulin. The present disclosure also relates to methods of augmenting tissue in humans, such as the skin or subcutaneous fat, comprising implanting or injecting such dermal fillers into the tissue.

Chronic ultraviolet radiation results in the accumulation of abnormal elastic fibers in the skin, termed “solar elastosis,” because they stain similar to elastin. These abnormal elastic fibers account for the majority of changes associated with an aged appearance in chronically sun-damaged skin including fine lines and wrinkles and sagging skin. Dramatic alterations of the superficial dermis are responsible for the deep wrinkles and laxity that occur in photodamaged skin. Immunohistochemical staining demonstrates that these poorly formed clumps of elastic fibers comprising solar elastosis are comprised of the normal constitutes of elastic fibers, elastin, fibrillin, versican, GAGs, and glycosemenoglycans.

One of the interesting features of solar elastosis, is that it is quite resistant to degradation, and persists throughout the lifetime of an individual. Most treatments directed at rejuvenating photodamaged skin cause an accumulation of new skin, including the previously mentioned “normal constitutes,” e.g., collagen, elastin, elastic fibers, and GAGs, in the very superficial dermis. Beneath this zone of normal-appearing skin, there still resides a significant amount of solar elastotic material despite even aggressive attempts at rejuvenating photodamaged skin. For example, even after carbon dioxide laser resurfacing, which removes not only the epidermis but also a portion of the superficial dermis entirely, there is still significant solar elastosis left in the dermis. This persistence of solar elastosis may be one of the biggest barriers to rejuvenating chronically sun-damaged skin. However, this persistence of solar elastotic material may also be seen as a potential benefit toward developing filler agents for humans and other mammals.

The objective of inserting a filler agent into an organ, including the skin, is to either fill an area in which there is currently a deficit of material which should normally be present, or to produce a structural change in an organ system. However, one problem that plagues the use of fillers in humans and other mammals is the digestion and or removal by other means of this dermal filler by the body. For instance, the human body reacts to foreign substances by producing an inflammatory response, and often removing or digesting the added material.

Photoaged skin currently treated aggressively with topical applications of anti-aging compounds, such as tretinoin or glycolic acid, dermabrasion, or laser resurfacing, the latter two of which remove the surface of the skin causing massive inflammation, all fail to remove 100% of the solar elastotic material. Thus, solar elastosis is significantly resistant to removal by an inflammatory response. This resistance to removal is one of the properties that would make solar elastosis an ideal filler material for the skin and other organ systems such as the esophageal and urinary tract sphincters, deficits in connective tissue, muscle or bone and other organs. In addition, various components of the elastic fibers are also variably resistant to digestion by proteases and other inflammatory mediators. These components would also seem to be ideal filler agents.

To address the foregoing problems while capturing the benefits of solar elastosis, the present disclosure is directed to dermal fillers for biomedical applications in humans and animals based on solar elastosis and/or other fillers based on or derived from elastic fibers. The present disclosure also relates to methods of tissue augmentation in mammals comprising implanting or injecting into the tissue of a mammal such dermal fillers.

In addition, the present disclosure is directed to a method of tissue augmentation in mammals comprising implanting or injecting into the tissue of a mammal at least one filler material. Non-limiting examples of the filler material that may be used include fibrillin, recombinant fibrillin, fibulin, and recombinant fibulin.

The present disclosure also relates to methods of augmenting the skin or subcutaneous fat of a human. These methods are particularly useful for treating common skin conditions, such as wrinkles, photoaging, acne scarring, scars, or HIV-associated lipoatrophy, and generally comprise implanting or injecting into the skin or subcutaneous fat of the human, the elastic material described above, including solar elastosis or the described fillers.

One aspect of the present disclosure is directed to functional implants for tissue augmentation or restoration in mammals. These functional implants can be used in a method of tissue augmentation in mammals comprising implanting or injecting into the tissue of a mammal elastic material derived from solar elastosis in mammals, such as humans.

As one skilled in the art would appreciate, the elastic material may be derived from the same human in which it is implanted. For example, although the dry weight of sun-protected skin is only about 4%, in sun-damaged skin, the elastosis itself is often the major extracellular matrix component present in the area of sun damage within skin. Photodamaged skin is often removed as a result of plastic surgery for a face lift in severely photodamaged individuals, making one's own solar elastosis available for implanting.

Cells taken from sun-damaged skin can also be grown in tissue culture, and elastic fiber components harvested. In addition, solar elastosis-like material, elastic fibers, or their components can be created in a laboratory either by a chemical reaction or by genetic engineering and would make this material readily available. Therefore, in one embodiment, the elastic tissue may be created ex-vivo to resemble in its properties solar elastosis.

In addition, cadaveric solar elastosis can be harvested from the skin of cadavers. Therefore, according to one embodiment of the present disclosure, the elastic material is derived from an outside source, such as a cadaver.

The present disclosure is also directed to a method of tissue augmentation in mammals comprising implanting or injecting into the tissue of a mammal at least one filler material. Non-limiting examples of the filler material that may be used include fibrillin, recombinant fibrillin, fibulin, and recombinant fibulin.

Fibrillin, a protein that is a component of elastic fibers is remarkably resistant to degradation. If one imagines a bungee cord representing an elastic fiber, with the stretchy part of the cord resembling elastin, the string on the outside that provides strength would be fibrillin. There have been a number of fibrillins described in the literature. Any fibrillin or combination of fibrillins would make an ideal filler material due to it's resistance to degradation and other properties.

According to one aspect of the disclosure, fibrillin is extracted from mammalian tissues or produced by genetic engineering using recombinant technologies. For example, in one embodiment recombinant fibrillin may be created in vitro using cells derived from various known sources, including Chinese hamster ovary cells, the milk of a transgenic animal, (such as goats, sheep, cows and mice), or a combination thereof.

In addition, elastic fibers harvested from mammalian sources could be treated to be more like solar elastosis and made resistant to degradation. Elastic fibers could also be treated with lysyl oxidase or lysyl oxidase-like proteins, such as lysyl oxidase-like 1, making them better function as an implant including making them longer lasting.

Fibrillin can also be derived from tissue, for example by digesting away the surrounding extracellular matrix of collagen and the elastin and proteoglycan components of elastic fibers as has been described in the medical literature, leaving the insoluable fibrillin behind.

Fibulin, a relatively newly described protein, interacts with elastic fibers and could also be used as a filler agent. Fibulin has been shown to associate with the fibrillin-rich fibers in the very superficial dermis of sun-damaged skin. Thus, any fibulin, alone or in combination with fibrillin or any other agents could be used as a filler. In fact, solar elastosis, elastic fibers treated to more closely resemble solar elastosis, fibrillin, or fibulin could be combined with other fillers or agents such as cytokine or growth factors that make these agents function better as fillers, either allowing them to last longer than they would alone or helping them to stimulate ingrowth of the mammals' own extracellular matrix.

The proteins fibrillin and fibulin can be produced by recombinant methods. These methods of preparation has the advantage of being free of most contaminants including other proteins and pathogens, and thus would be of high purity. Recombinant proteins can be made by a variety of methods including but not limited to using transgenic mammalian cells, bacteria, or transgenic animals that produce transgenic proteins in their blood or milk.

Any of the above-mentioned fillers can be used in combination with existing fillers such as but not limited to collagen, elastin, fibrillin, fibulin, decorin, biglycian, hyaluronic acid, calcium hydroxyapatite, silicone, cells, and poly L-lactic acid.

In one embodiment, chondroitin sulfate (CS) proteoglycan may be used as a filler material, either alone or co-distributed with other materials, such as elastic or collagen. As used hererin, the CS proteoglycans can encompass versican, decorin, or combination thereof.

It is understood that the tissue augmentation material, i.e., the dermal filler, may be combined in a suitable vehicle, which may include various active agents that achieve certain effects. For example, the vehicle may include one or more biologically active factors to aid in the healing, regrowth, stability or longevity of the natural tissue or to aid in the stability or longevity of the tissue augmentation material.

Nonlimiting examples of the biologically active factors include epidermal growth factor, heparin, transforming growth factor-beta, transforming growth factor-alpha, platelet-derived growth factor, basic fibroblast growth factor, connective tissue activating peptides, beta-thromboglobulin, insulin-like growth factor, interleukins, nerve growth factors, colonly stimulating factors, tumor necrosis factors, osteogenic factors, supernatant from tissue culture, bone morphogenic proteins, and combinations thereof.

Tissue augmentation materials, or fillers, can be used for a myriad of applications in mammals. One of the widest uses recently has been for cosmetic augmentation of skin for cosmetic or functional purposes. Recently a number of fillers have been approved for treating fine lines and wrinkles that result from aging, or more accurately photoaging. Fillers can be injected via a syringe intra-dermally to puff-up the skin resulting in diminution of the appearance of the wrinkle, fine line, or skin fold such as the nasolabial fold; or they may be inserted surgically through an incision in the skin. Fillers can also be used for augmenting loss of the fatty layer of skin as occurs commonly in HIV infected people.

In one aspect, the method, which are described herein may be used to augment the skin or subcutaneous fat of a human. This method is particularly useful for treating common skin conditions, such as wrinkles, photoaging, acne scarring, scars, or HIV-associated lipoatrophy. This method generally comprises implanting or injecting into the skin or subcutaneous fat of the human, elastic material described above, including solar elastosis and/or the described fillers. This same method can be used to prevent or reduce scarring of the skin of a human from various conditions, including those previously described.

In addition to cosmetic uses, fillers according to the present disclosure can be used to augment many tissues that have functional alterations that impair normal function or cause pain. For example, intervertebral discs can herniated and cause pain, resulting in numerous days of lost productivity from an individual and loss of quality of life due to pain. Filler materials may help in maintaining the proper intervertebral disc dimensions or prevent adhesions during surgery. A material that is not seen as foreign by the immune system or that is not easily digested away would be superior. Using such a filler to protect organ systems during any type of surgery from adhesions would be quite useful at preventing post-operative complications. Thus, agents that can prevent adhesions can also prevent scarring. The fact that solar elastosis is resistant to degradation may also confer the ability to prevent tissue reaction and thus prevent scarring and adhesions. These complications are common after my types of surgery including but not limited to abdominal procedures, orthopedic procedures and the like.

Fillers according to the present disclosure can also be used to augment the urethral sphincter in cases of urethral sphincter insufficiency. In addition, fillers can be used to augment the esophageal sphincter to help prevent acid reflux disease, which in turn can result in changes to the esophagus including Barratt's esophagus and possibly esophageal cancer. Fillers according to the present disclosure can also be used in dental practice to augment and support the teeth, bones and soft tissues of the mouth.

In one aspect of the present disclosure, there is provided a method of augmenting tissue or bone in or around the mouth by implanting or injecting the previously described materials into the bone or tissue in or around the mouth. As used herein, “in or around the mouth” is intended to encompass tissues and bones in the jaw, and/or mouth, such as the soft tissue in the mouth. This method can be used to treat mammals suffering from gum disease, such as gingivitis.

In addition, fillers according to the present disclosure can also be used to augment bone. Beneficially, the filler not only takes up space, but can also stimulate repair, serving as a provisional matrix for the in-growth of the surrounding structure such as skin or bone.

Therefore, other aspects of the present disclosure are directed to methods to augment other tissue in the body. Non-limiting examples of other tissues that can be augmented include the urinary bladder or the urinary bladder sphincter, bone, the esophageal sphincter, the teeth, dental ligaments, gums or other structures in the mouth.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Other embodiments of the invention will be apparent to those skilled in the art from consideration of the specification. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of tissue augmentation in mammals comprising implanting or injecting into the tissue of a mammal elastic material derived from solar elastosis or from at least one sun-damaged site in a mammal.

2. The method of claim 1, where the mammal is a human and the elastic material is derived from the same human in which it is implanted.

3. The method of claim 1, where the elastic material is derived from a cadaver.

4. The method of tissue augmentation in mammals comprising implanting or injecting into the tissue of a mammal elastic tissue created ex-vivo to resemble in its properties solar elastosis.

5. The method of tissue augmentation in mammals comprising implanting or injecting into the tissue of a mammal at least one filler material.

6. The method of claim 5, where the filler material is chosen from fibrillin, recombinant fibrillin, fibulin, and recombinant fibulin.

7. The method of claim 6, wherein the filler is recombinant fibrillin created in vitro using cells derived from Chinese hamster ovary cells, the milk of a transgenic animal, or a combination thereof.

8. The method of claim 7, wherein the transgenic animal is chosen from a goat, sheep, cow, and mouse.

9. The method of claim 1, wherein the material for tissue augmentation is combined with at least one additional tissue augmentation material chosen from collagen, elastin material, fibrillin, fibulin, decorin, biglycian, hyaluronic acid, calcium hydroxyapatite, silicone, cells, and poly L-lactic acid.

10. The method of claim 3, wherein the material for tissue augmentation is combined with at least one additional tissue augmentation material chosen from collagen, decorin, biglycian, hyaluronic acid, calcium hydroxyapatite, silicone, cells, and poly L-lactic acid.

11. The method of claim 4, wherein the material for tissue augmentation is combined with at least one additional tissue augmentation material chosen from collagen, elastin material, fibrillin, fibulin, decorin, biglycian, hyaluronic acid, calcium hydroxyapatite, silicone, and poly L-lactic acid.

12. The method of claim 5, wherein the material for tissue augmentation is combined with at least one additional tissue augmentation material chosen from collagen, solar elastosis, elastin material, fibulin, decorin, biglycian, hyaluronic acid, calcium hydroxyapatite, silicone, cells, and poly L-lactic acid.

13. The method of claim 1, 4, or 5, wherein the tissue augmentation material is combined in a suitable vehicle which further comprises one or more biologically active factors to aid in the healing, regrowth, stability or longevity of the natural tissue or to aid in the stability or longevity of the tissue augmentation material.

14. The method in claim 13, wherein the biologically active factors are chosen from epidermal growth factor, heparin, transforming growth factor-beta, transforming growth factor-alpha, platelet-derived growth factor, basic fibroblast growth factor, connective tissue activating peptides, beta-thromboglobulin, insulin-like growth factor, interleukins, nerve growth factors, colonly stimulating factors, tumor necrosis factors, osteogenic factors, supernatant from tissue culture, and bone morphogenic proteins.

15. The method of claim 1, 4, or 5, wherein the tissue to be augmented is the skin or subcutaneous fat of the mammal.

16. The method of claim 1, 4, or 5, wherein the tissue to be augmented is the urinary bladder or the urinary bladder sphincter.

17. The method of claim 1, 4, or 5, wherein the tissue to be augmented is bone.

18. The method of claim 1, 4, or 5, wherein the tissue to be augmented is the esophageal sphincter.

19. The method of claim 1, 4, or 5, wherein the tissue to be augmented is at least one of teeth, dental ligaments, gums, and other structures in the mouth.

20. A method of treating at least one condition chosen from wrinkles, photoaging, acne scarring, scars, or HIV-associated lipoatrophy, said method comprising implanting or injecting into the skin or subcutaneous fat of the human, elastic material derived from solar elastosis or from at least one sun-damaged site in a mammal.

21. A method for the prevention or reduction of scarring of the skin of a mammal, said method comprising implanting or injecting into the skin or subcutaneous fat of the mammal, elastic material derived from solar elastosis or or from at least one sun-damaged site in a mammal.

22. A method of augmenting human tissues that have functional alterations that impair normal function or cause pain, said method comprising implanting or injecting into the tissue of the human, elastic material derived from solar elastosis or from at least one sun-damaged site in a mammal.

23. The method of claim 22, said method comprising implanting or injecting into the esophageal sphincter of a human suffering from acid reflux elastic material derived from solar elastosis or from at least one sun-damaged site in a mammal.

24. The method of claim 22, said method comprising implanting or injecting into the intervertebral discs of a mammal suffering from a herniated disc material derived from solar elastosis or from at least one sun-damaged site in a mammal.

25. The method of claim 22, said method comprising implanting or injecting into bone or tissue in or around the mouth material derived from solar elastosis or from at least one sun-damaged site in a mammal.
